(19) United States
(12) Patent Application Publication
(10) Pub. No.: US 2005/0273109 A1
(43) Pub. Date: Dec. 8, 2005

(54) Computer controlled reaming device

(75) Inventor: Todd M. Bjork, River Falls, WI (US)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(73) Assignee: Minnesota Scientific, Inc., St. Paul, MN (US)

(21) Appl. No.: 10/858,579

(22) Filed: Jun. 2, 2004

(51) Int. Cl. A61B 17/16

(52) U.S. Cl. 606/80; 606/79

(57) ABSTRACT

An apparatus reams a cavity into a bone. The apparatus includes a cutting tool that is operably attached to a drive. A controller is in communication with the drive wherein the controller manipulates movement of the drive and the cutting tool in three dimensions such that the cutting tool reams a precise cavity into the bone.

### BACKGROUND OF THE INVENTION

The present invention relates to a computer controlled reaming device. In particular, the present invention relates to a computer controlled reaming device that is useful in reaming a cavity of a selected size and configuration into a bone.

Humans experience joint pain due to damage to the joint, typically caused traumatic injury, arthritis or because of wear. Joint pain most commonly occurs in the elderly due to arthritis and wear over time. To alleviate pain caused by damaged joints, joint replacement surgeries have become more commonly performed.

Two common joint replacement surgeries are hip replacement surgery and shoulder replacement surgery. In the shoulder replacement surgery, the glenoid cavity of the shoulder joint is reamed or enlarged to accept an insert. Similarly, in the hip replacement surgery the acetabulum is reamed or enlarged to accept the insert. The insert is typically manufactured of polyethylene.

The insert includes a cavity which accepts either the femoral ball of the hip joint or the humoral ball of the shoulder joint. Alternatively, either the humoral ball or femoral ball can be surgically removed and replaced with a metal spherical head that is inserted into the polyethylene insert.

The insert is typically secured in the reamed cavity with a cement. Besides securing the insert into the cavity, the cement also compensates for imperfections created in the cavity reamed into the bone during the reaming process by filling in gaps between the insert and the reamed bone.

A significant amount of torque can be exerted upon the joints of the body during daily activities as well as during strenuous activities. Ideally, the insert and the bone withstand the torque while the cement's primary purpose is to retain the insert in a selected position within the cavity. However, when the cement is required to fill in voids between the bone and the insert, the cement must also withstand the torque exerted upon the joint. Over time, the cement may not be able to withstand the torque which may cause the cement to fail. When the cement fails, the insert within the bone loosens and causes discomfort and pain.

### SUMMARY OF THE INVENTION

The present invention includes an apparatus for reaming a cavity into a bone. The apparatus includes a cutting tool that is operably attached to a drive. A controller is in communication with the drive wherein the controller manipulates movement of the drive and the cutting tool in three dimensions such that the cutting tool reams a precise cavity into the bone.

### BRIEF DESCRIPTION OF THE DRAWINGS

FIG. 1 is a perspective view of the computer controlled reaming device of the present invention.

FIG. 2 is a side view of the computer controlled reaming device of the present invention positioned above a surgical site.

FIG. 3 is a flow diagram of a control scheme of the present invention.

### DETAILED DESCRIPTION OF THE PREFERRED EMBODIMENTS

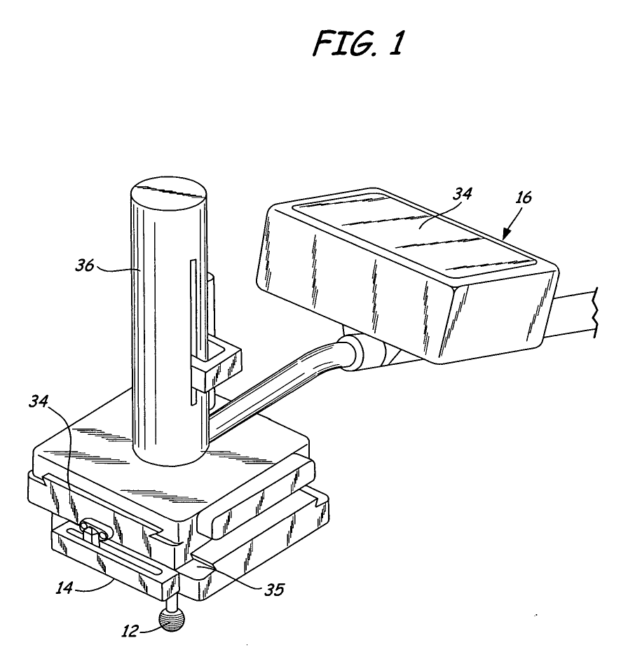

A computer controlled reaming device of the present invention is generally depicted at 10 in FIG. 1. The reaming device 10 includes a cutting tool 12 that is operably attached to a drive 14 that moves in three dimensions and is controlled by a programmable computer 16. The device 10 is designed to ream a cavity of a predetermined size, depth, and configuration into a bone.

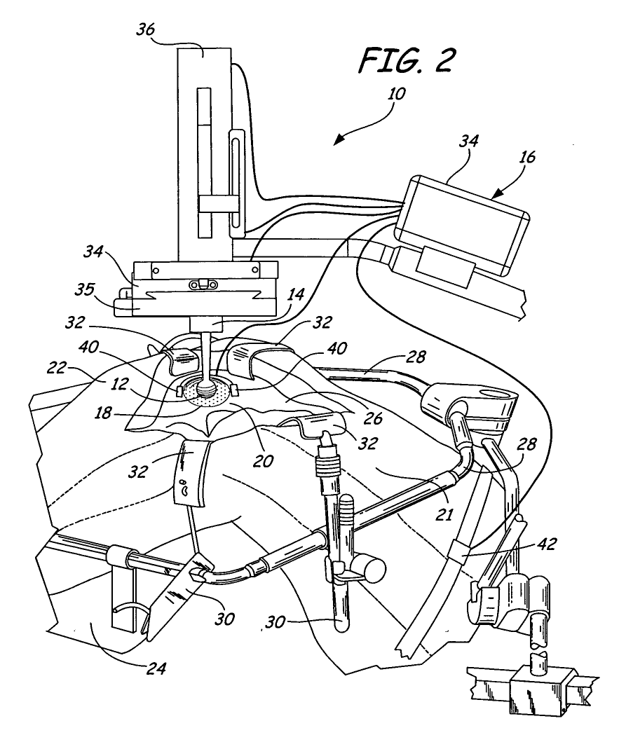

The computer controlled reaming device 10 is particularly useful in precisely reaming an acetabulum 18 of a hip joint 20 as illustrated in FIG. 2. The computer controlled reaming device 10 is also useful in reaming a glenoid cavity (not shown) of a shoulder joint (not shown) to accept an insert (not shown) with minimal reaming error. The computer controlled reaming device 10 is also useful in other surgical procedures such as preparing a bone for the insertion of a pin which is used to aid in mending a break or any other surgical procedure that requires a bone to be reamed. Going forward, reference will be made to the hip joint and the acetabulum although the computer controlled reaming device 10 can also be used to ream cavities into other joints or bones.

When preparing for a hip replacement surgery, an insert is selected for insertion into the acetabulum 18 based upon the size, age, and condition of the joint of the patient. The size, shape and configuration of the insert is programmed into the programmable computer 16. Preferably, the computer 16 includes a preprogrammed catalog of the specifications of the available inserts such that the operator may select the size, shape and configuration of the cavity to be reamed into the acetabulum prior to the surgery by selecting the insert, preferably from a touch screen 34.

With the insert selected, the patient 22 is positioned on an operating table 24 and a surgical wound 26 is made into the patient 22. A table mounted retractor support apparatus 28 is positioned about the surgical wound 26. Retractors 30 are positioned on the retractor support apparatus 28 where retractor blades 32 are inserted into the surgical wound 26 and retract flesh to expose the hip joint 20. With the hip joint 20 exposed, the femeral ball is dislocated from the acetabulum 18, preferably utilizing method disclosed in U.S. Pat. No. 6,368,271 for retracting the femoral ball. Alternatively the method disclosed in U.S. Pat. No. 6,315,718 is preferably used to retract a humoral ball when shoulder replacement surgery is performed. U.S. Pat. Nos. 6,315,718 and 6,368,271 are hereby incorporated by reference in their entireties.

Referring only to the hip replacement surgery, the femoral ball is dislocated from the acetabulum 18, and the computer controlled reaming device 10 is positioned about the surgical wound 26 such that the cutting tool 12 is positioned within the acetabulum 18. The operator selects the program associated with the insert where the program includes size, shape and configuration of the cavity to be reamed into the acetabulum 18 corresponding to the size, shape and configuration of the preselected insert such as a partial spherical shape. Preferably, a touch screen 34 is used to select the program associated with the selected insert, although other user interfaces such as a keyboard, a mouse and a joystick are within the scope of the invention.

Referring to FIGS. 1-3, the computer program controls the movement of the drive 14 in three dimensions and also controls a supply of power to the drive 14. Any apparatus that is controllable to move an object in three dimensions can be used to practice the invention. Preferably, the drive 10 includes a V-groove system 34, 35 to move the cutting tool 12 in a horizontal plane and an electromagnetic cylinder 36 to move the cutting tool 12 in the vertical plane. Both the V-groove systems 34, 35 and the cylinder 36 are powered by electromagnetic force, although electric, pneumatic and hydraulic drives are within the scope of the invention

The computer 16 controls the movement of the drive 14 and the cutting tool 12 to ream a cavity corresponding to the outer size and shape and configuration of the insert. While the computer 16 controls the movement of the drive 14 and the cutting tool 12, the computer 16 cannot determine the relative position of the drive 14 and the cutting tool 12 relative to the acetabulum 18. One or more sensors 40, which senses the position of the cutting tool 12 and the drive 14 relative to the acetabulum 18, are positioned proximate the surgical site. Preferably, a plurality of sensors 40 are positioned about the surgical site to detect the position of the cutting tool 12 and the drive 14 relative to the acetabulum 18. The sensors 40 provide feedback to the computer 16.

When the sensors 40 detect misalignment of the cutting tool 12 relative to the acetabulum 18, the sensors 40 send a signal to the computer 16 to adjust for the misalignment. Alternatively, the sensors 40 may send a signal to the computer 16 to shut down the cutting tool 12 and the drive 14 to prevent the cutting tool 12 from reaming a misaligned cavity into the acetabulum 18.

A sensor 42 or a plurality of sensors can also be positioned on the leg 21 of the patient 22 to detect movement of the patient 22 while the cutting tool 12 reams the acetabulum 18. The sensors 42 communicate with the computer 16 to detect movement and when movement outside of a margin of error is detected the computer 16 shuts down power to the drive 14 and the cutting tool 12. With the drive 14 and the cutting tool 12 shut down, the acetabulum 18 is realigned with the cutting tool 12 and the drive 14 such that the precise cavity will be reamed into the acetabulum 18. With the cutting tool 12 and the drive 14 realigned with the acetabulum 18, the user restarts the computer program to continue reaming the cavity.

With the cavity corresponding to the computer program reamed into the acetabulum 18, the surgeon positions the insert into the cavity to determine the fit of the insert within the cavity. With a proper fit between the acetabulum 18 and the insert, the surgeon applies a layer of adhesive or cement to the insert and fixedly secures the insert within the cavity. With the insert secured within the acetabulum 18, the femoral ball is reinserted into the insert and the surgical wound 26 is sutured.

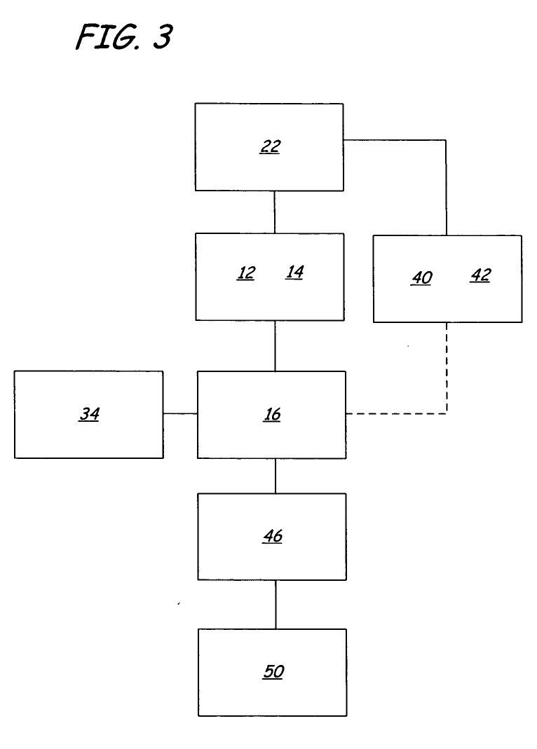

If the surgeon determines that the insert does not fit properly within the acetabulum 18, the computer 16 includes an option to expand the cavity, while maintaining the same configuration. Referring to FIG. 3, the program is placed into a manual mode 46 and the surgeon manually inputs the increase in the size. The surgeon places the program into the automatic mode and the cavity is reamed to include the inputted increased dimensions.

Besides reaming the cavity at a speed determined by the computer 16, the surgeon can control the speed at which the cavity is reamed by selecting the manual mode 46 on the computer 16. In the manual mode 46 the computer controls the size, shape and configuration of the acetabulum 18. However, the surgeon decides the speed at which the drive 14 and the cutting tool 12 operate. Also, in the manual mode 46 the surgeon determines the order in which the acetabulum 18 is reamed. Preferably, the computer controlled reaming device 10 includes a tracing pad 50 for the surgeon to manually control the position of the cutting tool 12 and the drive 14, although a mouse, a joystick or a keyboard may also be used to control the drive 14 with the cutting tool 12.

Although the present invention has been described with reference to preferred embodiments, workers skilled in the art will recognize that changes may be made in form and detail without departing from the spirit and scope of the invention.

1. An apparatus for reaming a cavity into a bone, the apparatus comprising:
a bone cutting tool;
a drive wherein the bone cutting tool operably attaches to the drive; and
a controller in communication with the drive and wherein the controller manipulates the movement of the drive in three dimensions such that the drive moves and rotates the cutting tool to ream the cavity into the bone.

2. The apparatus of claim 1 and wherein the drive comprises an electric powered drive.

3. The apparatus of claim 1 and wherein the drive comprises a pneumatic powered drive.

4. The apparatus of claim 1 and wherein the drive comprises a hydraulic powered drive.

5. The apparatus of claim 1 and wherein the controller comprises a programmable computer.

6. The apparatus of claim 5 and wherein the programmable computer includes a program for a geometric shape and dimensions of the cavity and wherein the controller controls the drive and the cutting tool to form the cavity to have the programmed geometric shape and dimensions.

7. The apparatus of claim 1 and wherein the computer comprises an automatic control wherein the computer controls the movement of the drive and the cutting tool.

8. The apparatus of claim 1 and wherein the computer comprises a manual bypass mode wherein a user controls the movement of the drive and the cutting tool.

9. The apparatus of claim 8 and wherein the controller further comprises a tracing pad wherein when in the manual bypass mode the user controls the movement of the drive and the cutting tool with the tracing pad.

10. The apparatus of claim 1 and wherein the apparatus reams a partial spherical shaped cavity into an acetabulum.

11. A method of implanting a device into a bone of a patient, the method comprising:
making a surgical wound in the patient;
creating a surgical site through the surgical wound to expose the bone into which the device is to be implanted;
selecting an insert to be implanted into the bone;
determining an exterior three dimensional size of the device;
programming the exterior three dimensional size of the device into a controller;
positioning a reaming device comprising a driver and a cutting tool attached to the driver proximate the bone;
controlling the driver and the cutting tool with the controller such that the reaming device reams a cavity into the bone corresponding to the exterior three dimensional configuration of the device; and
implanting the device into the cavity.

12. The method of claim 11 and further comprising pre-programming a plurality of three dimensional configurations corresponding to selected three dimensional exterior configurations of devices into the controller.

13. The method of claim 12 and wherein one of the three dimensional configurations is selected from a touch-screen controller.

14. A computer controlled bone reaming device comprising:
a drive device;
a reaming tool connected to the drive device; and
a computer communicating with the drive device wherein the computer controls movement of the drive device and the reaming tool such that a pre-programmed three dimensional cavity programmed into the computer is reamed into the bone for precisely implanting a device having a complementary configuration to a surface of the cavity.

15. The device of claim 14 and wherein the computer further comprises a plurality of three dimensional cavity programs wherein the user selects the three dimensional cavity to be reamed without having to reprogram the computer.

16. The device of claim 14 and further comprising a touch control screen wherein the pre-programmed three dimensional cavity is selected from the touch control screen.

17. The device of claim 14 and wherein the computer further comprises a manual mode such that a surgeon can manually operate the computer to ream the cavity into the bone.

18. The device of claim 14 and wherein the bone to be reamed is the pelvis such that an acetabulum is reamed to accept a hip replacement device.
